(19) United States
(12) Patent Application Publication
(10) Pub. No.: US 2021/0228161 A1
(43) Pub. Date: Jul. 29, 2021

(54) Tissue hemoglobin measuring instrument and tomographic reconstruction method for oxyhemoglobin/deoxyhemoglobin concentrations

(71) Applicant: Wellness Allied Inc, Natick, MA (US)

(72) Inventors: Weihui Li, Natick, MA (US); Tao ZHANG, Natick, MA (US)

(21) Appl. No.: 17/205,229

(22) Filed: Mar. 18, 2021

(51) Int. Cl. A61B 5/00; A61B 5/1455; A61B 5/0205; G16H 10/40; G16H 50/50

(52) U.S. Cl. CPC A61B 5/742; A61B 5/14552; A61B 5/7225; A61B 5/02427; G16H 10/40; G16H 50/50; A61B 5/0205

(57) ABSTRACT

A measurement system and calculation method for the concentration distribution of oxyhemoglobin and deoxyhemoglobin in human tissues are provided. The measurement system includes an array of hemoglobin sensor modules, a control system, a signal analysis device and a display system. The hemoglobin sensor module includes a light source transmitter and a photoelectric sensor, which are used to emit and receive light respectively. When the control system triggers the hemoglobin sensor module to emit light, it controls multiple photoelectric sensors to receive the emitted light and upload relevant data. The signal analysis system evaluates the tissue blood oxygen concentration inside human tissues based on the emitted light, and proposes a tomographic three-dimensional reconstruction method. Based on the hemoglobin measurement system and detection method, this patent provides a low-cost, easy-to-use evaluation method for evaluating the blood circulation health level of human tissues.

### TECHNICAL FIELD

The present invention generally relates to a tissue hemoglobin measuring instrument in the medical field, which is used to measure the concentration of deoxyhemoglobin and oxyhemoglobin in tissue through tomographic reconstruction, and to evaluate the blood circulation conditions based on the measurement results.

### BACKGROUND

The human body activities rely on the cardiovascular system to supply nutrients and oxygen to each cell, and to take away metabolic waste. When the blood circulation is compromised, the cells and tissues in the affected area will lack of nutrients and oxygen, resulting functional or organic disorders. In Chinese Medicine, this is called blood stasis and the treatment methods include acupuncture, bloodletting, herbal prescription, massage, and moxibustion.

There is a problem, however, for the Chinese medicine to treat blood stasis: there is no medical devices that can locate the blood stasis location quickly. With the existing technologies, the options for non-invasive evaluation of blood circulation include: pulse oximeter to measure arterial oxygen saturation level (SpO2), near infrared spectroscopy (NIRS) to measure an averaged number of tissue oxygen saturation level (StO2) and diffuse reflectance correlation spectrometer for blood flow measurement (also called diffusion correlation spectroscopy).

Pulse oximeter is mainly based on two physical principles: (a) Measurable pulsatile arterial signal, which is relatively independent of non-pulsatile arteries, veins, capillaries and other tissues; (b) Oxyhemoglobin (HbO2) and deoxyhemoglobin (Hb) have different absorption spectrum. Currently available pulse oximeters use two light-emitting diodes (LEDs) to emit red and infrared wavelengths. The two specific wavelengths are used because HbO2 and Hb have different absorption coefficients for these two wavelengths. In the red region, HbO2 absorbs less light than Hb, while in the infrared region, the HbO2 absorbs more. According to different light absorption coefficient, pulse oximeter can estimate oxygen saturation, as well as the pulse waves.

The near-infrared spectroscopy (NIRS) is also based on the attenuation of near-infrared light (600-1000 nm wavelength) transmitted in human tissues. Current NIRS usually has one light source and two light detectors. The distance between the light incident point and the exiting point determines the size of the tissue sample. The NIRS signal mainly comes from the small blood vessels (arterioles, capillaries, and venules) present in the tissue sample. With different absorption coefficient of oxyhemoglobin and deoxyhemoglobin to light, the oxyhemoglobin and deoxyhemoglobin concentrations can be calculated. For details, please refer to Owen-Reece's review [1]. The NIRS system can also estimate the amount of hemoglobin contained in the sampling area, and display it in total tissue hemoglobin index (HbT) or absolute tissue hemoglobin index (THI). Currently, the measurement of tissue oxygen is a dynamic process that tracks the changes in tissue oxygen over time. It is generally used to monitor the blood circulation status of patients during surgery or to monitor critically ill patients in the intensive care unit.

The existing NIRS system has three measurement modes: time domain (TD), frequency domain (FD) and continuous wave (CW). Although the TD-NIRS measurement system has the advantage of relatively rich measurement information, it is expensive, and requires longer measurement time, which make it difficult to use and generate continuous time series in clinic. The FD-NIRS system usually needs an above 200 MHz frequency modulation to achieve a reasonable signal-to-noise ratio and to measure the phase shift. It is not practical to perform multi-point continuous measurement in clinic. Therefore, CW system is the mainstream technology of optical imaging. Based on CW technology, scientists build diffuse optical tomography system with multiple NIRS. Diffuse optical tomography has been under development for more than 30 years, and the difficulties are due to the fact that light is strongly scattered and the diffusive photons are used for image reconstruction. The algorithm is based on diffusion equation inverse problems, requiring high computation load. Also, diffusion equation is invalid in low-scattering and/or highly absorbing regions and in the vicinity of light sources. The inverse problem is inherently ill-posed and highly underdetermined. The above reasons explain why diffuse optical tomography hasn't been used in clinic practice yet [2].

The above-mentioned pulse oximeter technology has generated many patents. Usually, the patents make use of two LEDs and one photodiode to measure oxygen saturation. There are also patents that use more luminous light sources, and multiple light detectors to form measurement array and measure the oxygen saturation in pulsatile arteries. U.S. Pat. No. 8,798,702B2 has one or more light sources and multiple light detectors and uses a multiplexer to sequentially select each of the light detectors, instead of measuring the emitted light simultaneously. Such a system cannot dynamically observe the multi-point pulse and oxygen saturation.

When come to the NIRS technology to measure tissue oxygen saturation, there are the following types of patents that have been applied for or granted: 1) Use of a NIRS for the measurement of head blood oxygen saturation (WO2016210357A1, US20180116525A1, CN2691489Y, CN107252305A, US20160345880A1); 2) Use NIRS to detect the concentration of photosensitive substances and blood oxygen saturation in tissues (CN103610467B); 3) Use a functional NIRS to monitor the mental state of the brain (U.S. Pat. No. 9,848,812); 4) Use light source array and photoelectric sensor array to measure tissue oxygen saturation (US20160081603A1).

Patent applications WO2016210357A1 and US20180116525A1 use light-emitting diodes and photodiode, or their arrays. However, this patent does not have a detailed description of the control circuit and how to calculate oxygen saturation. The application has not yet been granted a patent.

CN2691489Y applied an utility model patent in China to monitor blood oxygen concentration in the brain. This patent mainly designs an interface circuit for data sampling, data processing and a digital-to-analog conversion circuit.

The tissue blood oxygen saturation monitor patented by CN103610467B has a detailed design of light source array and photoelectric sensor array. Each pair of light source and photoelectric sensor is parallel, and the photoelectric sensor only detects light signal emitted by the paired light source.

CN107252305A uses square wave to modulate LED frequency, which excites multi-channel, multi-wavelength phase-locked photon counter to detect signals in a fully parallel mode. Based on this mode, the applicant of the patent built the functional NIRS brain imaging system. The light in the system is introduced into the brain tissue through optical fibers. Its detection system also uses optical fiber to transmit the emitted light to photomultiplier tube. Using of optical fiber makes the system inconvenient to use and expensive.

The main contribution of U.S. Pat. No. 9,848,812 is to perform noise cleaning and data analysis for the emitted light signal and evaluate the mental state of the measured subjects. The patent also proposed sensor arrays, but did not specifically present the array control method and blood oxygen concentration calculation method.

The patent, US20160081603A1, proposed a reflective light source array and photoelectric sensor array to detect blood oxygen in human tissues, but did not gave specific implementation method, and the patent application was finally abandoned.

The main invention of US20160345880A1 is to propose a system that can measure the blood oxygen saturation of brain tissue and the blood oxygen metabolism of brain tissue. The system uses laser and optical fiber for light source and cannot be applied to clinic conveniently.

Regarding the diffuse optical tomography system, the U.S. Pat. No. 7,242,997B2 proposed a non-contact diffuse optical tomography system, which uses a laser light source and requires a complex device structure, which cannot be safely and conveniently used in the clinic. The patent of U.S. Pat. No. 9,861,319B2 also proposed a non-contact diffuse optical tomography that uses laser as a light source and uses optical lenses, photoelectric sensors or charge-coupled device to measure emitted light. The complex structure and high price only allow its use in experimental research. It is impractical for clinical use at this moment.

U.S. Pat. No. 9,545,223B2 uses optical fiber array for light source in the measurement of tissue oxygen concentration in the brain. The fact that the light source array emit light to the brain tissue simultaneously will interfere with each other and generate noise to the signal. Also, use of optical fibers as light-emitting sources result in poor portability and high cost. The patent is currently used for blood oxygen detection in brain tissue.

U.S. Pat. No. 7,983,740B2 designs multiple light-emitting light sources, multiple photoelectric detectors, and each light-emitting light source is individually controlled to avoid interference. Each photoelectric detector has a separate analog-to-digital converter, which improves its dynamic bandwidth. However, its independent light-emitting light source and photoelectric detector are not integrated and makes the system relatively big. Also, the patent did not present tomographic reconstruction algorithm.

### SUMMARY

Based on the analysis of the previous technologies, this patent proposes a hemoglobin distribution measurement system for human tissues, including: a hemoglobin sensor module array, each hemoglobin sensor module includes: a light source, used to emit light of one or more frequencies and transmit the light to human tissue; a light detector, used to receive reflected light from human tissue and generate corresponding electric signals; a control circuit, used to coordinate light sources and the light detectors: the control circuit generates a control signal to enable a light source to emit light; at the same time, the control circuit triggers all or some of the light detectors that are within a predetermined distance from the enabled light source to detect reflected light from the human tissue; the control circuit repeats the previous two steps until all light sources have been enabled sequentially; a signal analysis system, configured to: receiving the output electrical signals from the light detectors; performing tomographic reconstruction of oxyhemoglobin and deoxyhemoglobin concentration distribution; a display system, used to display the concentration distribution of oxyhemoglobin and deoxyhemoglobin.

Optionally, the hemoglobin sensor module is an integrated chip with each light source and each light detector forming a pair and being arranged adjacent, and each light source has a standard luminous intensity and luminous time.

Optionally, each hemoglobin sensor module has independent filter, amplifier and analog-to-digital converter.

Optionally, the control system communicates with the hemoglobin sensor module through I2C or SPI data bus to sequentially enable the light source of each hemoglobin sensor module and to upload corresponding outputs from light detectors.

Optionally, the light source of a hemoglobin sensor module emits light, the control system use GPIO ports to trigger all or part of light detectors within a predetermined distance from the active light source to simultaneously detect the reflected light.

Optionally, the measurement system further comprises a plurality of hemoglobin sensor module arrays, with each hemoglobin sensor module array is integrated together, but is physically separated from other hemoglobin sensor module arrays, to measure hemoglobin concentrations at different body parts.

Optionally, the measurement system further comprises analysis of signal qualities from light detectors, and selecting high quality signals for tomographic reconstruction, wherein the indicators for signal quality include: the absolute intensity of the reflected light, the ratio of the reflected light intensity to the incident light intensity, the signal-to-noise ratio of the reflected light, and/or the stability of the signal in the time or frequency domain.

Optionally, the carrier for the hemoglobin sensor module array is a flexible and opaque material.

Optionally, the predetermined distance is 2-20 cm.

Optionally, each hemoglobin sensor module also measures blood oxygen saturation, pulse wave and heart rate for the body tissue directly underneath the hemoglobin sensor module.

Optionally, the measurement system further comprises constructing a two-dimensional pulse wave propagation graph based on the pulse waves measured by hemoglobin sensor modules, the positional information of the hemoglobin sensor modules, and the measuring time.

Optionally, the measurement system further comprises evaluation the healthy level of blood circulation in human tissues based on the oxyhemoglobin and deoxyhemoglobin concentration distribution, pulse wave and/or pulse oxygen saturation.

According to another aspect of the present invention, a method for tomographic reconstruction of oxyhemoglobin and deoxyhemoglobin concentration distribution in human tissues is provided, which includes: establishing a three-dimensional model for the human tissue where the hemoglobin sensor module array is located; defining the oxyhemoglobin and deoxyhemoglobin concentration variables on the three-dimensional model; establishing light propagation path model for each light source to each light detector; deriving variable equations for the oxyhemoglobin and deoxyhemoglobin concentration at discrete points on the light propagation path through interpolation of defined oxyhemoglobin and deoxyhemoglobin concentration variables; controlling each light source in the hemoglobin sensor module array is to emit light in turn, and simultaneously controlling all or part of light detectors within a predetermined distance from the active light source to measure reflected light at different positions; writing light attenuation equations for all or part of the light detectors responding to each light source, wherein, the equations comprising the following parameters or variables: incident light intensity, light propagation path information, oxyhemoglobin and deoxyhemoglobin concentration variables on the light propagation path, light absorption coefficients by oxyhemoglobin and deoxyhemoglobin, and reflected light intensity; solving light attenuation equation set to obtain the concentration distribution of oxyhemoglobin and deoxyhemoglobin.

Optionally, the three-dimensional model of human tissue is a finite element model, with finite element being tetrahedron, pentahedron or hexahedron; the concentration variables of oxyhemoglobin and deoxyhemoglobin are defined at each vertex of the finite element.

Optionally, the light propagation path model from each light source to each light detector is a smooth curve.

Optionally, the light propagation path model from each light source to each light detector is a half-ellipse shape, with semiminor axis d being light incidence depth, and major axis L being distance between the light source and the light detector, the semiminor axis d is 1/N of the major axis L, and N is a positive number greater than 1.

Optionally, the propagation path model from each light source to each light detector is banana-shaped.

Optionally, the inner and outer curvatures of the banana-shaped propagation path is defined by two semi-ellipses with a common major axis and a common ellipse center, but the length of semiminor axis are different.

Optionally, cross sections of the banana-shaped light propagation path along ellipse radiuses are circles, with the diameters of the circles being the differences of the corresponding radiuses of these two half-ellipses.

Optionally, the light attenuation equation is:

Io/I=exp{Σᵢ₌₁^{M}((a1*Hbᵢ(x,y,z)+a2*HbOᵢ(x,y,z))*ΔLᵢ)},

where Iₒ is the intensity of the incident light, I is the intensity of the reflected light, M means that the light propagation path is divided into M segments, ΔLᵢ is the length of the i-th segment on the light propagation path, and Hbᵢ(x,y,z) is deoxyhemoglobin concentration of the i-th segment on the light propagation path, HbOᵢ(x,y,z) is oxyhemoglobin concentration of the i-th path on the light propagation path, and a₁ and a₂ are the molar attenuation coefficients of deoxyhemoglobin and oxyhemoglobin, respectively.

Optionally, the light propagates along a banana-shaped path, and the light attenuation equation is:

Iₒ/I=exp{Σᵢ₌₁^{M}((a1*Hbᵢ(x,y,z)+a2*HbOᵢ(x,y,z))*ΔLᵢ*r*ΔSᵢ)}

where Iₒ is the intensity of the incident light, I is the intensity of the reflected light, M means that the light propagation path is divided into M segments, with each segment being approximately a truncated cone, Hbᵢ(x,y,z) is the deoxyhemoglobin concentration of the i-th segment on the light propagation path, HbOᵢ(x,y,z) is the oxyhemoglobin concentration of the i-th segment on the light propagation path, a1 and a2 are the molar attenuation coefficients of deoxyhemoglobin and oxyhemoglobin, respectively, and ΔLᵢ is the height of the truncated cone for the i-th segment on the light propagation path, ΔS is the cross-section area of the i-th segment on the light propagation path in the middle, and r is a diffuse reflection path coefficient to be optimized through experiments.

Optionally, deoxyhemoglobin and oxyhemoglobin concentration on light propagation path are interpolated from the deoxyhemoglobin and oxyhemoglobin concentration variables at the vertices of the finite elements through the finite element interpolation basis function.

According to another aspect of the present invention, a method for evaluating the blood circulation health level of human tissues is provided, which includes: controlling the light source on the hemoglobin sensor module to emit light of one or more frequencies and transmit the light waves to the patient's tissue; controlling multiple light detectors at different positions to simultaneously detect the reflected light from human tissue and generating corresponding output signals; determining the signal quality of the output signals and selecting high quality signals to perform tomographic reconstruction and calculate oxyhemoglobin and deoxyhemoglobin concentrations, and/or calculating pulse wave and oxygen saturation from each hemoglobin sensor module, and also calculating the oxygen saturation distribution and/or pulse wave propagation path based with the hemoglobin sensor module array positional information; assessing the blood circulation health condition based on the concentration distribution of oxyhemoglobin, deoxyhemoglobin, pulse wave propagation pattern and/or the oxygen saturation map.

In the present invention, hemoglobin sensor modules are used to build a hemoglobin sensor module array. The module has standard luminous intensity and luminous time, which lead to small error in the measurement results. The device is also convenient to use, and has a very low cost. The control of this system adopts I2C or SPI bus for data communication, which can control each light source to emit light in turn, and can simultaneously control multiple light detectors through the GPIO port to simultaneously detect the reflected light from the human tissue, which increases the sampling frequency of the measurement. The present invention also provides a tomographic reconstruction method, which is used to calculate the 3D concentration distribution of oxyhemoglobin and deoxyhemoglobin based on the light attenuation equations. Meanwhile, each hemoglobin sensor module can measure pulse wave and blood oxygen saturation individually and can calculate out a two-dimensional pulse wave propagation path and two-dimensional blood oxygen saturation distribution underneath the array. According to the 3D concentration distribution of oxyhemoglobin and deoxyhemoglobin, the propagation path of pulse wave and the 2D distribution of blood oxygen saturation, the healthy level of blood circulation in human tissues can be accurately assessed.

### BRIEF DESCRIPTION OF THE DRAWINGS

FIG. 1. shows the absorption coefficients of various molecules in human tissues to incident light. The figure is adopted from prior literature [3].

FIG. 2A. shows a design diagram of a measurement system to measure oxyhemoglobin and deoxyhemoglobin concentrations in human tissues.

FIG. 2B shows a schematic diagram of a light source 250 emitting light and light detectors receiving reflected light according to an embodiment of the present invention, and the dotted lines represent the propagation paths of lights.

FIG. 3 shows a three-dimensional model for the human tissue according to an embodiment of the present invention, which is composed of hexahedral finite element elements.

FIG. 4 shows a structural design diagram of a system for measuring oxyhemoglobin and deoxyhemoglobin distribution in human tissues according to an embodiment of the present invention. 450 is a hemoglobin sensor module composed of LED (light source), photodiode (light detector), amplifier, filter, and digital-to-analog converter (D/A converter). 440 is a LED enable selector, or a multiplexer, to enable one LED to emit one frequency light at each moment.

FIG. 5 shows another system for measuring oxyhemoglobin and deoxyhemoglobin distribution in human tissues according to an embodiment of the present invention. This system is composed of two hemoglobin sensor module arrays to facilitate symmetrical measurement of the left and right sides of the human body.

FIGS. 6A and 6B show a schematic diagram of pulse waves and an isochronous diagram of pulse propagation according to an embodiment of the present invention.

FIG. 7 shows a three-dimensional distribution diagram of blood stasis obtained after tomographic reconstruction according to an embodiment of the present invention.

FIG. 8 schematically shows a two-dimensional diagram of pulse oxygen saturation according to an embodiment of the present invention.

FIG. 9 shows a schematic diagram of the diffuse reflection of light from a light source to a light detector described in the previous document [4]. The light propagation follows a banana-shaped path.

FIG. 10 shows a schematic diagram of the light propagation path models for tomographic reconstruction. FIG. 10A shows a model when the light propagation path is a half ellipse; FIG. 10B shows a model when the light propagation path is in a banana-shape.

FIG. 11 shows the light detectors with optimal distances from light sources. The optimal distance is bigger than m and smaller than n, as shown in the line-dotted region.

FIG. 12 shows an implementation process of an embodiment of the present invention.

FIG. 13A shows an interpolation method using hexahedron finite element as an example.

FIG. 13B shows how to calculate natural coordinate system ξ₁, ξ₂, and ξ₃ based on the mapping between a hexahedron finite element and the unit cube with known (x, y, z) and (xⱼ, yⱼ, zⱼ).

### DETAILED DESCRIPTION

The specific embodiments of the present invention will be described below in conjunction with the drawings.

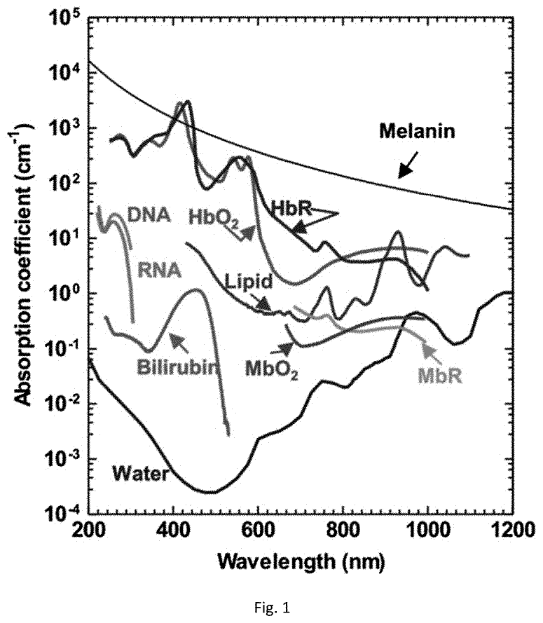

FIG. 1 Shows the incident light absorption coefficients of various molecules in human tissues. Different molecules have different absorption coefficients for incident light. Based on this fact, the concentration of human oxyhemoglobin and deoxyhemoglobin can be estimated by experiment designing, model development, and calculations.

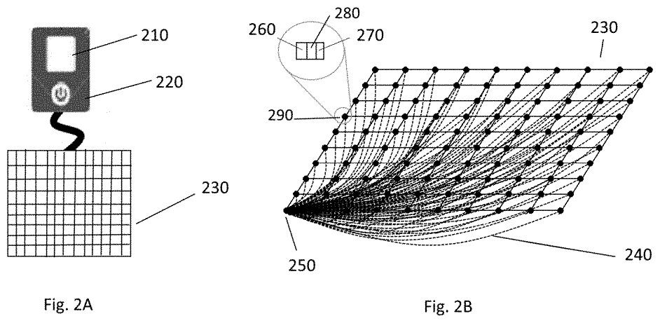

FIG. 2A shows a design diagram of a measurement system to measure oxyhemoglobin and deoxyhemoglobin concentrations in human tissues according to an embodiment of the present invention, including a controlling end 220 and a measuring end 230, which are connected to each other. The controlling end also has a display system 210. There are buttons on the surface of the controlling end for human manipulation, and there are controlling circuit and a signal analysis part inside the control end. The controlling circuit is used to coordinate the light sources and the light detectors: the controlling circuit generates a controlling signal to enable a light source to emit light; at the same time, the controlling circuit triggers all or part of the light detectors that are within a predetermined distance from the enabled light source to detect reflected light from the human tissue; repeats the previous two steps until all light sources have been enabled sequentially. The signal analysis part receives the output signals from light detectors and determines the signal qualities of the output signals; calculates oxyhemoglobin and deoxyhemoglobin concentrations through tomographic reconstruction.

FIG. 2B shows a schematic diagram of a light source emitting light and all light detectors receiving light according to an embodiment of the present invention. 230 is the measuring end, and each black dot represents one hemoglobin sensor module. 250 and 290 are two hemoglobin sensor modules. In this example, each hemoglobin sensor module consists of two LEDs, shown as 260 and 280, and one photodiode, shown as 270 in the enlarged view of the black dot hemoglobin sensor module. The wavelengths of the light waves emitted by the LEDs are between 620 and 1000 nm. In this image, one of the 250's LEDs is emitting light and all the photodiodes are receiving light. 240 is one path of light propagation. During detection, the controlling circuit controls each LED to emit light sequentially, and the light emitting time can be on the order of 20 microseconds (us) to several milliseconds (ms). All photodiodes can receive light at the same time.

As mentioned above, the Near Infrared Spectroscopy (NIRS) for tissue oxygen saturation measurement in the prior arts usually have a single light source and two light detectors to calculate a tissue oxygen value. In contrast, the hemoglobin tomography system for oxyhemoglobin and deoxyhemoglobin of human tissues in the embodiment of the present invention emits one light wave a time by one light source, and multiple detectors receive light simultaneously. From there, the system can then obtains a set of equations. By solving the set of equations, the oxyhemoglobin and deoxyhemoglobin concentrations of the segmented units of human tissue are obtained.

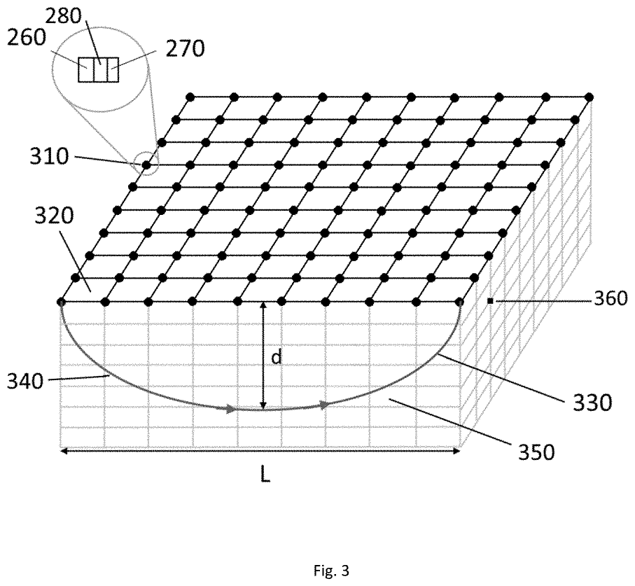

FIG. 3 shows a human tissue region segmentation model for tomographic reconstruction according to an embodiment of the present invention. In the array, the LED emits light of two different wavelengths in sequence, with wavelength range between 600 and 1000 nm.

The attenuation of light through human tissue is described by the Beer-Lambert formula, which is expressed in the form:

ln(Io/I)=a·c·L

Or

Io/I=exp(a·c·L)

where, Io is the incident light intensity and I is the reflected exiting light intensity. a is the molar attenuation coefficient. c is the concentration of the substance that absorbs light, and L is the length of the light path.

When light passes through a path in human tissue having one substance of different concentrations, the final attenuation formula of light can be expressed as:

Io/I=exp(∫a·cᵢ(x,y,z)dl)º

Or it can be expressed in discrete form:

Io/I=exp(Σa·cᵢ(x,y,z)Δl).

where cᵢ(x,y,z) represents the concentration of the light-absorbing substance with coordinates (x,y,z) at the i-th segment on the path, and ΔL represents the length of the i-th segment on the path through which light passes.

When light passes through a space with two light-absorbing substances of non-uniform concentrations, the attenuation formula can be expressed as:

Io/I=exp(Σ(a₁c₁ᵢ(x,y,z)+a₂c₂ᵢ(x,y,z)),Δl)

In the figure, 310 is one hemoglobin sensor module, including two light-emitting diodes (260, 280) and a light detector (270). 320 shows a hexahedron finite element for the human tissue segmentation model. 330 is the reflected light, and 340 is the incident light. In this example, the reflected light and the incident light form a half ellipse, with the semiminor axis d and the major axis length L. 350 is the human tissue through which light passes. We can write an equation for light absorbance from one LED to one light detector based on the elliptical path, the oxyhemoglobin and deoxyhemoglobin concentrations on the path and the incident, reflected light intensities. Each light source in the array emits light sequentially, and is received by multiple light detectors. An equation set can be written. Through matrix calculation, the oxyhemoglobin and deoxyhemoglobin concentrations can be obtained.

In the above example, the segmentation model of the human tissue are hexahedron finite elements. The segmentation model can be other shapes, such as tetrahedron, pentahedron. The oxyhemoglobin and deoxyhemoglobin concentrations are defined on finite element vertices.

In the above example, the light path in the human tissue is set as a half-ellipse shape, the distance L between the light source and the light detector defines the major axis of the ellipse, the semiminor axis defines the light incidence depth. In the model, semiminor axis d equals ⅓ of L. This is a preferred option, and other ranges can be set. Preferably, the semiminor axis d is set to be ¼ to ½ of the major axis L, and more preferably the semiminor axis d is 1/3.5 to 1/2.5 of the major axis L.

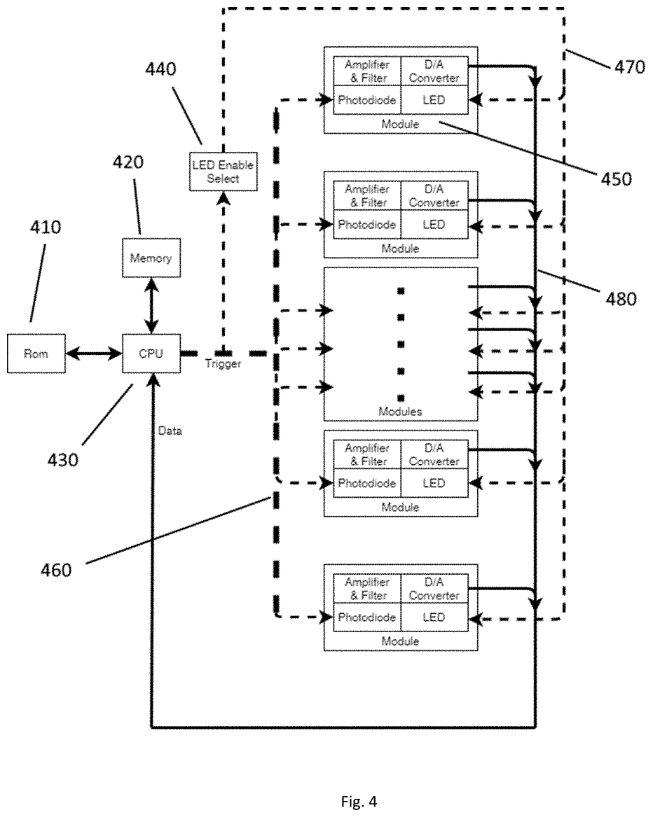

FIG. 4 shows a structural design diagram of a system for detecting oxyhemoglobin and deoxyhemoglobin concentrations in human tissues according to an embodiment of the present invention. 450 is a hemoglobin sensor module composed of light source (LED), light detector (photodiode), amplifier, filter, and digital-to-analog converter. In this example, the light source emitter is LED. To use the system, a hemoglobin sensor module array is attached to the surface of the human body. 440 is a multiplexer, used to select light sources. Each time period only one light source emits light of one frequency. 470 is the data bus to select light sources. 460 is the controlling data bus, which is used to control part or all of the light detectors to simultaneously receive lights from the light source. 480 represents data bus for data transmission. 430 is the central processing unit that controls this system. 410 and 420 are memories for storing programs and data. The multiplexer 440, the central processing system 430, and the memories are usually arranged at the controlling end for operation. Some of them can also be hosted on cloud computers. When the system is running, the central processing unit sends out a control signal to select a light source to emit light of a frequency through the multiplexer. At the same time, a control signal triggers part or all light detectors to receive the light emitted by the light source and reflected by human tissue. After the light detectors receiving the reflected lights, the signals are processed by the amplifiers, filters, and analog-to-digital converters of hemoglobin sensor modules 450. The processed digital signals are transmitted to the central processing unit 430 through the data bus 480. In the central processing unit 430, the oxyhemoglobin and deoxyhemoglobin concentrations are computed through tomographic reconstruction, the pulse oxygen saturation and pulse propagation are calculated. The signal processing and tomographic reconstruction can also be done in cloud computers.

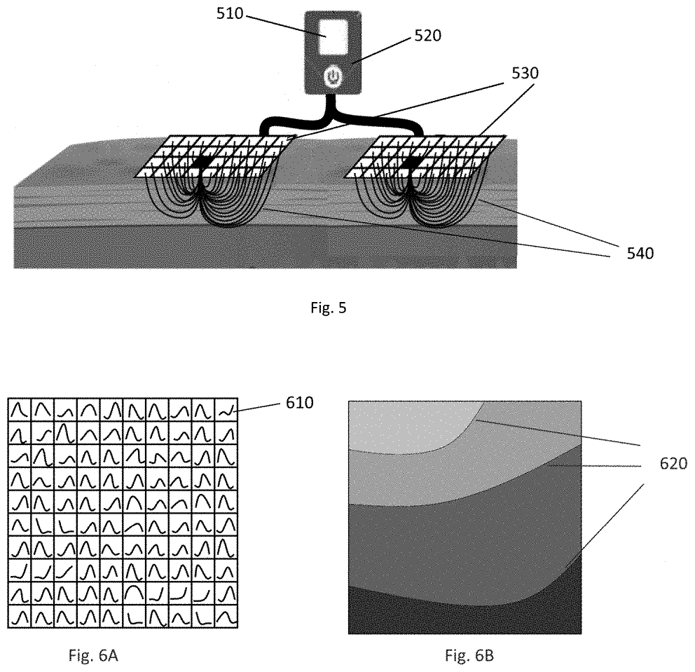

FIG. 5 shows a tissue hemoglobin measuring instrument composed of two measurement arrays according to an embodiment of the present invention. This system can be conveniently used to measure the tissue blood oxygen, pulse wave and pulse blood oxygen saturation distribution symmetrically for human tissues, which can better find blood circulation problems of the human body. 510 is a display system, 520 is a controlling end, 530 are two measurement arrays, and 540 is are light paths.

FIG. 6A shows a two-dimensional pulse waves according to an embodiment of the present invention. The pulse wave shown in each small square in the figure is calculated by each hemoglobin sensor module. FIG. 6B shows a pulse wave propagation diagram based on the occurring timing of the pulse wave characteristic points. The pulse wave characteristic point can be the maximum ascending slope point, or the pulse wave peak. 620 is the isochronous line of the pulse wave mark points. The diagram is color coded, indicating time sequence of the pulse wave marking points, from which we can see the wave propagation direction.

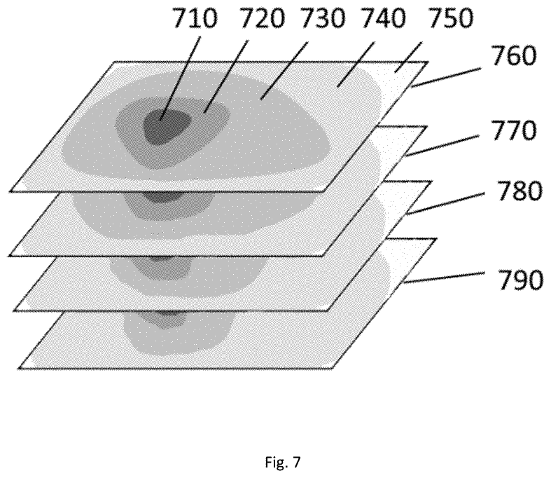

FIG. 7 schematically shows a three-dimensional distribution diagram of blood stasis obtained after tomographic reconstruction according to an embodiment of the present invention. According to an embodiment of the present invention, a method for calculating the degree of blood stasis is: the degree of blood stasis is proportional to the ratio of deoxyhemoglobin to the sum of deoxyhemoglobin and oxyhemoglobin: deoxyhemoglobin content/(oxyhemoglobin concentration+deoxyhemoglobin concentration). Another method for estimating the degree of blood stasis is based on the tissue pulse wave velocity: the pulse wave velocity is inversely proportional to blood stasis. Another method to estimate the degree of blood stasis is to calculate the weighted average of tissue pulse wave velocity, the tissue oxygen saturation. FIG. 7 only shows the distribution of blood stasis in 4 layers of tissue (760, 770, 780, 790). In practice, there can be more or fewer layers. The numbers 710, 720, 730, 740, 750 in FIG. 7 indicate different degrees of blood stasis in different areas of a layer. The colors of the areas gradually become lighter, indicating that the degree of blood stasis gradually decreases.

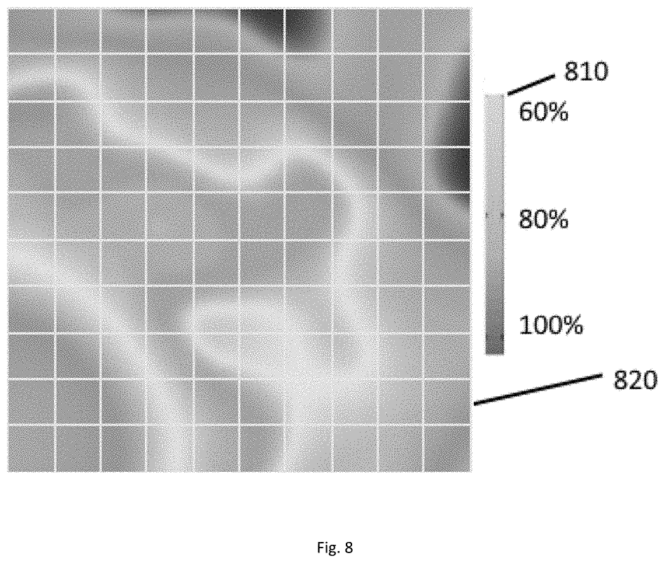

FIG. 8 shows an example of a two-dimensional distribution of pulse oxygen saturation according to an embodiment of the present invention. Some values in the two-dimensional distribution are obtained by interpolation of values measured by each hemoglobin sensor module. 810 is a color scale, marking pulse oxygen saturation percentage. 820 is the two-dimensional distribution of pulse oximetry.

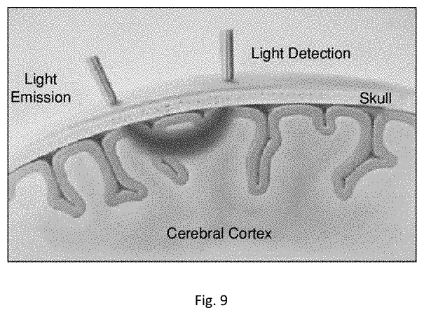

FIG. 9 shows a schematic diagram of the diffuse reflection of light from the light source to the light detector described in the previous documents [4]. The light propagation follows a banana-shaped path.

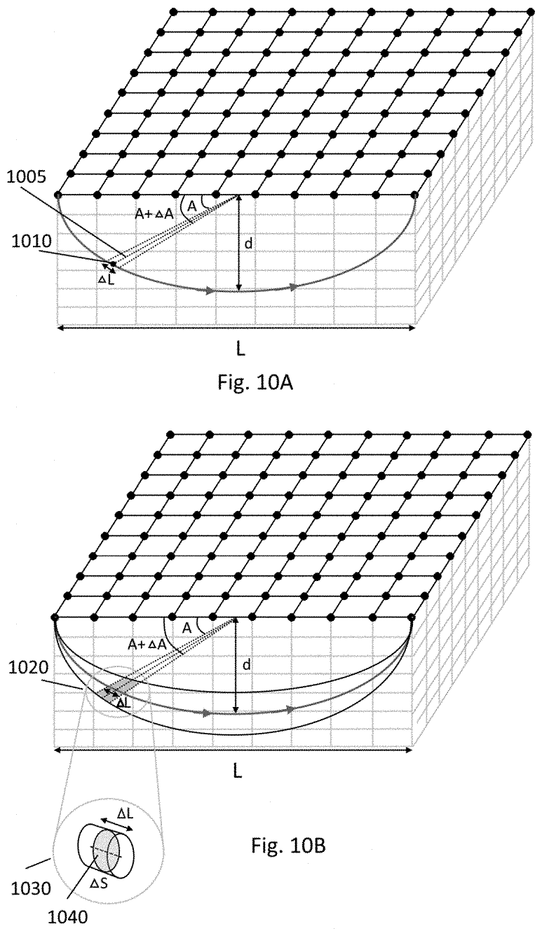

FIG. 10A shows a schematic diagram of a mathematical model established for tomographic reconstruction for calculating oxyhemoglobin and deoxyhemoglobin concentration according to an embodiment of the present invention. The model of FIG. 10A does not consider light diffusion, and the propagation path of light is a semi-ellipse. In the figure, the i-th segment on the path has an angle of A and A+ΔA. 1005 is the angle bisector of the angle, and the coordinates of intersection 1010 of the angle bisector and the ellipse is (x, y, z). The light attenuation equation is:

Io/I=exp{Σᵢ₌₁^{M}((a1*Hbᵢ(x,y,z)+a2*HbOᵢ(x,y,z))*ΔLᵢ)},

where Io is the intensity of the incident light, I is the intensity of the reflected light, M indicates that the light propagation path is divided into M segments, ΔLᵢ is the length of the i-th segment on the light propagation path, a1 and a2 are molar attenuation coefficients of deoxyhemoglobin and oxyhemoglobin respectively. Hbᵢ(x,y,z) is the deoxyhemoglobin concentration of the i-th segment on the light propagation path. HbOᵢ(x,y,z) is the oxyhemoglobin concentration of the i-th segment on the light propagation path. Hbᵢ(x,y,z) and HbOᵢ(x,y,z) are computed out through interpolation from oxyhemoglobin and deoxyhemoglobin concentrations defined on the finite element vertices, based on finite element interpolation basis functions.

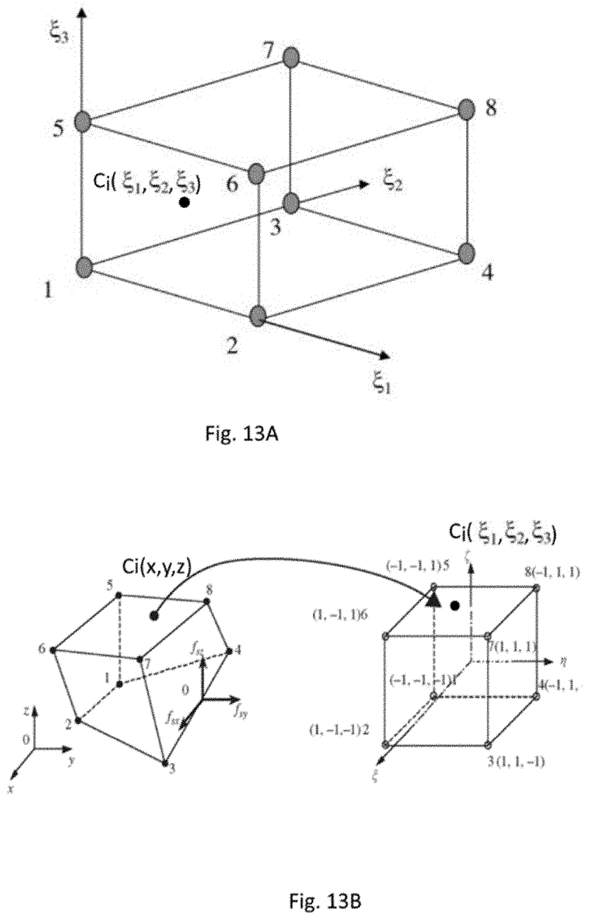

FIG. 13A shows an interpolation method using hexahedron finite element as an example. We define the concentrations of substance that absorbs light at vertex 1 to vertex 8 as: c1 to c8. Ci(x,y,z) is the concentration at any coordinates within a finite element element, and its interpolation formula is:

$$Ci ( x , y , z ) = {\sum }_{j = 1}^{8} {ψ}_{j} ( {ξ}_{1} , {ξ}_{2} , {ξ}_{3} ) * {c}_{j}$$

where

ψ₁(ξ₁,ξ₂ξ₃)=φ₁(ξ₁)φ₁(ξ₂)φ₁(ξ₃)

ψ₂(ξ₁,ξ₂ξ₃)=φ₂(ξ₁)φ₁(ξ₂)φ₁(ξ₃)

ψ₃(ξ₁,ξ₂ξ₃)=φ₁(ξ₁)φ₂(ξ₂)φ₁(ξ₃)

ψ₄(ξ₁,ξ₂ξ₃)=φ₂(ξ₁)φ₂(ξ₂)φ₁(ξ₃)

ψ₅(ξ₁,ξ₂ξ₃)=φ₁(ξ₁)φ₁(ξ₂)φ₂(ξ₃)

ψ₆(ξ₁,ξ₂ξ₃)=φ₂(ξ₁)φ₁(ξ₂)φ₂(ξ₃)

ψ₇(ξ₁,ξ₂ξ₃)=φ₁(ξ₁)φ₂(ξ₂)φ₂(ξ₃)

ψ₈(ξ₁,ξ₂ξ₃)=φ₂(ξ₁)φ₂(ξ₂)φ₂(ξ₃)

φ₁(ξ)=1−ξ

φ₂(ξ)=ξ

where ξ₁, ξ₂, and ξ₃ are natural coordinate system in a unit cube, and can be calculated out based on the mapping between a hexahedron finite element and the unit cube with known (x, y, z) and (xⱼ, yⱼ, zⱼ) (FIG. 13B).

$$x = {\sum }_{j = 1}^{8} {N}_{j} ( {ξ}_{1} , {ξ}_{2} , {ξ}_{3} ) * {x}_{j}$$

$$y = {\sum }_{j = 1}^{8} {N}_{j} ( {ξ}_{1} , {ξ}_{2} , {ξ}_{3} ) * {y}_{j}$$

$$z = {\sum }_{j = 1}^{8} {N}_{j} ( {ξ}_{1} , {ξ}_{2} , {ξ}_{3} ) * {z}_{j}$$

where

$${N}_{1} = \frac{1}{8} ( 1 - {ξ}_{1} ) ( 1 - {ξ}_{2} ) ( 1 - {ξ}_{3} ) \\ {N}_{2} = \frac{1}{8} ( 1 + {ξ}_{1} ) ( 1 - {ξ}_{2} ) ( 1 - {ξ}_{3} ) \\ {N}_{3} = \frac{1}{8} ( 1 + {ξ}_{1} ) ( 1 + {ξ}_{2} ) ( 1 - {ξ}_{3} ) \\ {N}_{4} = \frac{1}{8} ( 1 - {ξ}_{1} ) ( 1 + {ξ}_{2} ) ( 1 - {ξ}_{3} ) \\ {N}_{5} = \frac{1}{8} ( 1 - {ξ}_{1} ) ( 1 - {ξ}_{2} ) ( 1 + {ξ}_{3} ) \\ {N}_{6} = \frac{1}{8} ( 1 + {ξ}_{1} ) ( 1 - {ξ}_{2} ) ( 1 + {ξ}_{3} ) \\ {N}_{7} = \frac{1}{8} ( 1 + {ξ}_{1} ) ( 1 + {ξ}_{2} ) ( 1 + {ξ}_{3} ) \\ {N}_{8} = \frac{1}{8} ( 1 - {ξ}_{1} ) ( 1 + {ξ}_{2} ) ( 1 + {ξ}_{3} )$$

FIG. 10B shows a schematic diagram of a mathematical model established for tomographic reconstruction for calculating oxyhemoglobin and deoxyhemoglobin concentration according to an embodiment of the present invention. This model considers the diffusion of light, and the propagation path of light is banana-shaped. In this example, two curves (1040) of the banana-shaped propagation path are defined by two semi-ellipses with a common major axis and a common ellipse center, but different semiminor axis length. The banana-shaped path has a circular cross-section shown in the enlarged diagram 1030. The extension of the cross-section passes through the ellipses' center. The diameter is the difference of these two ellipse axis length. Light travels along a banana-shaped path, and the resulting light attenuation equation is:

Io/I=exp{Σᵢ₌₁^{M}(((a1*Hbᵢ(x,y,z)+a2*HbOᵢ(x,y,z))*ΔLᵢ*r*ΔSᵢ}

where Io is the intensity of the incident light, I is the intensity of the reflected light, M indicates that the light propagation path is divided into M segments, and each segment is approximately a truncated cone. Hbᵢ(x,y,z) is deoxyhemoglobin concentration of the i-th segment on the light propagation path. HbOᵢ(x,y,z) is the oxyhemoglobin concentration of the i-th segment on the light propagation path. a1 and a2 are the molar attenuation coefficients of deoxyhemoglobin and oxyhemoglobin, respectively, and ΔLᵢ is the height of the i-th section, ΔSᵢ is the average cross-section area of the i-th section on the light propagation path, and r is the diffuse reflection path coefficient, which is estimated by the actual length when diffuse reflection is considered and approximate length when the diffuse reflection is not considered.

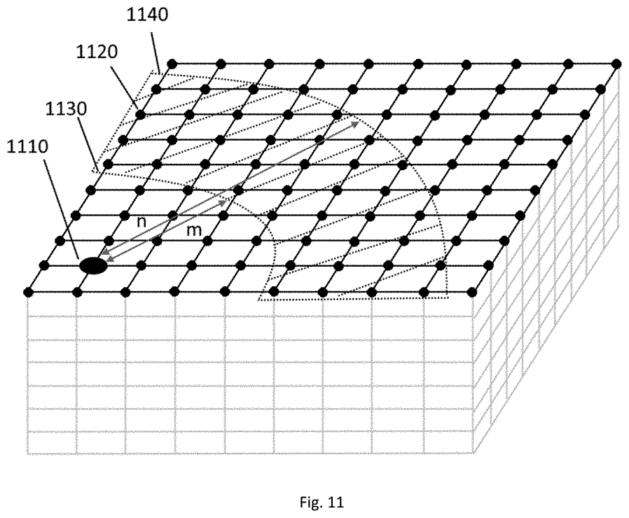

Preferably, generally a good signal can be detected if the distance between the incident point and the reflected point is in the range of 2-20 cm. More preferably, a better signal can be detected if the incident point to the reflected point is in the range of 2-6 cm. FIG. 11 shows a dotted-line shaded area. The hemoglobin sensor modules in the shaded area between the dashed lines of 1130 and 1140 meet the distance preferences of m-n cm from the light source 1110. The reflected light from each light source will be detected by light detectors according to the rule of 2-6 cm. If there are N light sources and the number of light detectors for the i-th light source is nᵢ, then the total number of equations is Σₙ₌₁^{N}nᵢ. The number of equations also determine the maximum number of finite elements that can be generate during tomographic reconstruction.

In summary, according to an embodiment of the present invention, a tomographic reconstruction method to find the concentration distribution of oxyhemoglobin and deoxyhemoglobin in human tissues is provided, which includes: establishing a three-dimensional model for the human tissue where the hemoglobin sensor array is located; defining the oxyhemoglobin and deoxyhemoglobin concentration variables on the three-dimensional model; defining light propagation path for each light source to each light detector; expressing the oxyhemoglobin and deoxyhemoglobin concentrations for discrete points on the propagation path with above defined variables through interpolation; controlling each light source in the hemoglobin sensor array to emit light in sequence and to measure the reflected light with the light detectors that are within a predetermined range from the selected light source; writing light attenuation equations from light sources to light detectors according to the light propagation path, the oxyhemoglobin and deoxyhemoglobin concentration variables on the light path, absorption coefficient of the light and the incident/reflected light intensity; solving the light attenuation equation set to get the concentration distribution of oxyhemoglobin and deoxyhemoglobin.

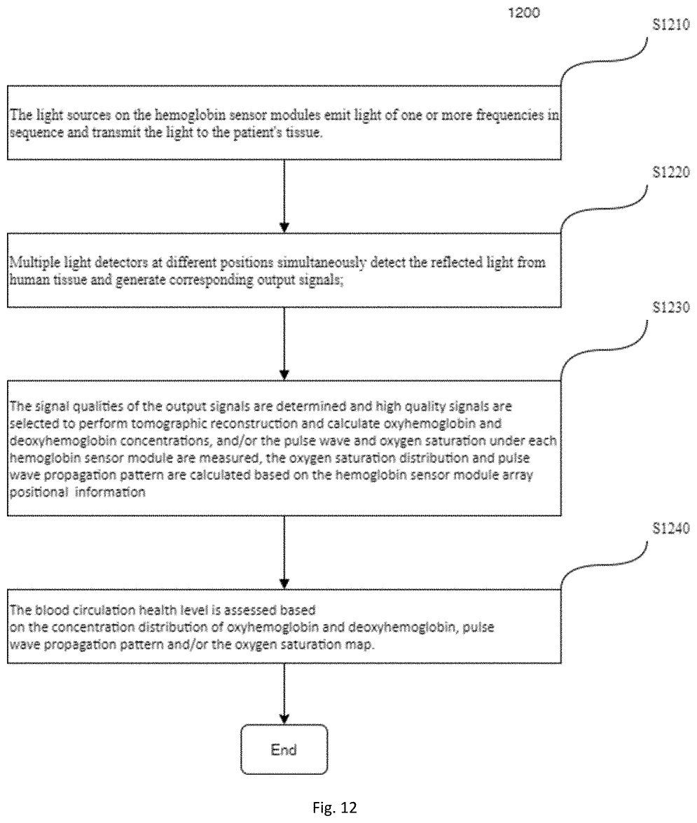

FIG. 12 shows an overall flowchart of a method 1200 for evaluating human tissue blood circulation health level.

In S1210, the light sources on the hemoglobin sensor modules emit light of one or more frequencies in sequence and transmit the light to the patient's tissue;

In S1220, multiple light detectors at different positions simultaneously detect the reflected light from human tissue and generate corresponding output signals;

In S1230, the signal qualities of the output signals are determined and high quality signals are selected to perform tomographic reconstruction and calculate oxyhemoglobin and deoxyhemoglobin concentrations, and/or the pulse wave and oxygen saturation under each hemoglobin sensor module are measured, the oxygen saturation distribution and pulse wave propagation pattern are calculated based on the hemoglobin sensor module array positional information;

In S1240, the blood circulation health level is assessed based on the concentration distribution of oxyhemoglobin and deoxyhemoglobin, pulse wave propagation pattern and/or the oxygen saturation map.

The hemoglobin measurement system and method can be used to guide acupuncture, moxibustion, massage and/or cupping. The measurement system has one or more of the following advantages:

(1) By activating the light sources in sequence, and controlling multiple light detectors to measure the reflected light after the human tissue absorption, a light attenuation equation set can be written and solved. Thus oxyhemoglobin and deoxyhemoglobin concentration distribution in human tissue can be calculated simultaneously. The results reveal more physiological and pathological information when compare to the averaged oxyhemoglobin and deoxyhemoglobin concentration obtained by single NIRS system.

(2) Compared with the prior art of three-dimensional reconstruction system using optical fibers as the light sources and light detectors, the embodiment of the present invention uses integrated chips of the hemoglobin sensor module. Therefore, the cost is reduced from tens of thousands of dollars in the prior art to less than $1,000, which will significantly benefit the marketing of hemoglobin testing products.

The various embodiments of the present invention have been described above, and the above description is exemplary, not exhaustive, and is not limited to the disclosed embodiments. Without departing from the scope and spirit of the illustrated embodiments, many modifications and changes are obvious to those of ordinary skill in the art. Therefore, the protection scope of the present invention should be subject to the protection scope of the claims.

1. A measurement system to measure oxyhemoglobin and deoxyhemoglobin concentrations in human tissues, including:
a hemoglobin sensor module array on a carrier, each hemoglobin sensor module includes: a light source, used to emit light of one or more frequencies and transmit the light to human tissue; a light detector, used to receive reflected light from human tissue and generate corresponding electric signals;
a control circuit, used to coordinate light sources and the light detectors: the control circuit generates a control signal to enable a light source to emit light; at the same time, the control circuit triggers all or some of the light detectors that are within a predetermined distance from the enabled light source to detect reflected light from the human tissue; the control circuit repeats the previous two steps until all light sources have been enabled sequentially;
a signal analysis system, configured to: receiving the electrical signals from the light detectors; performing tomographic reconstruction of concentration distribution of oxyhemoglobin and deoxyhemoglobin based on the electrical signals;
a display system, used to display the concentration distribution of oxyhemoglobin and deoxyhemoglobin.

2. The measurement system of claim 1, wherein the hemoglobin sensor module is an integrated chip with each light source and each light detector forming a pair and being arranged adjacent, and each light source has a same fixed luminous intensity and luminous time.

3. The measurement system of claim 1, wherein each hemoglobin sensor module has independent filter, amplifier and analog-to-digital converter.

4. The measurement system of claim 1, wherein the control system communicates with the hemoglobin sensor module through I2C or SPI data bus to sequentially enable the light source of each hemoglobin sensor module and to send corresponding outputs from light detectors to the signal analysis system.

5. The measurement system of claim 1, when the light source of a hemoglobin sensor module emits light, the control system use GPIO ports to trigger all or part of light detectors within a predetermined distance from the active light source to simultaneously detect the reflected light.

6. The measurement system of claim 1, comprising a plurality of hemoglobin sensor module arrays, with each hemoglobin sensor module array is integrated together, but is physically separated from other hemoglobin sensor module arrays, to measure hemoglobin concentrations at different body parts.

7. The measurement system of claim 1, further comprising analysis of signal qualities from light detectors, and selecting high quality signals for tomographic reconstruction, wherein indicators for signal quality include: absolute intensity of the reflected light, ratio of the reflected light intensity to the incident light intensity, signal-to-noise ratio of the reflected light, and/or stability of the signal in time or frequency domain.

8. The measurement system of claim 1, wherein a carrier for the hemoglobin sensor module array is of flexible and opaque material.

9. The measurement system according to claim 1, wherein the predetermined distance is 2-20 cm.

10. According to the measurement system of claim 1, each hemoglobin sensor module also measures blood oxygen saturation, pulse wave and heart rate for the body tissue directly underneath the hemoglobin sensor module.

11. The measurement system of claim 10, further comprising constructing a two-dimensional pulse wave propagation graph based on the pulse waves measured by hemoglobin sensor modules, positional information of the hemoglobin sensor modules, and measuring times.

12. The measurement system of claim 1, further comprising evaluating healthy level of blood circulation in human tissues based on the oxyhemoglobin and deoxyhemoglobin concentration distribution, pulse wave and/or pulse oxygen saturation.

13. A method for tomographic reconstruction of oxyhemoglobin and deoxyhemoglobin concentration distribution in human tissues, including:
establishing a three-dimensional model for the human tissue where the hemoglobin sensor module array is located;
defining the oxyhemoglobin and deoxyhemoglobin concentration variables on the three-dimensional model;
establishing light propagation path model for each light source to each light detector;
deriving variable equations for the oxyhemoglobin and deoxyhemoglobin concentration at discrete points on the light propagation path through interpolation of defined oxyhemoglobin and deoxyhemoglobin concentration variables;
controlling each light source in the hemoglobin sensor module array is to emit light in turn, and simultaneously controlling all or part of light detectors within a predetermined distance from the active light source to measure reflected light at different positions;
writing light attenuation equations for all or part of the light detectors responding to each light source, wherein, the equations comprising the following parameters or variables: incident light intensity, light propagation path information, oxyhemoglobin and deoxyhemoglobin concentration variables on the light propagation path, light absorption coefficients by oxyhemoglobin and deoxyhemoglobin, and reflected light intensity;
solving light attenuation equation set to obtain the concentration distribution of oxyhemoglobin and deoxyhemoglobin.

14. The method of claim 13, the three-dimensional model of human tissue is a finite element model, with finite element being tetrahedron, pentahedron or hexahedron; the concentration variables of oxyhemoglobin and deoxyhemoglobin are defined at each vertex of the finite element.

15. The method of claim 13, the light propagation path model from each light source to each light detector is a smooth curve.

16. The method of claim 13, the light propagation path model from each light source to each light detector is a half-ellipse shape, with semiminor axis d being light incidence depth, and major axis L being distance between the light source and the light detector, the semiminor axis d is 1/N of the major axis L, and N is a positive number greater than 1.

17. The method of claim 13, the propagation path model from each light source to each light detector is banana-shaped.

18. The method of claim 17, inner and outer curvatures of the banana-shaped propagation path are defined by two semi-ellipses with a common major axis and a common ellipse center, but the length of semiminor axis are different.

19. The method of claim 18, cross sections of the banana-shaped light propagation path along ellipse radiuses are circles, with the diameters of the circles being the differences of the corresponding radiuses of these two half-ellipses.

20. The method of claim 13, the light attenuation equation is:
Io/I=exp{Σᵢ₌₁^{M}((a1*Hbᵢ(x,y,z)+a2*HbOᵢ(x,y,z))*ΔLᵢ)},
where Iₒ is the intensity of the incident light, I is the intensity of the reflected light, M means that the light propagation path is divided into M segments, ΔLᵢ is the length of the i-th segment on the light propagation path, and Hbᵢ(x,y,z) is deoxyhemoglobin concentration of the i-th segment on the light propagation path, HbOᵢ(x,y,z) is oxyhemoglobin concentration of the i-th path on the light propagation path, and a₁ and a₂ are the molar attenuation coefficients of deoxyhemoglobin and oxyhemoglobin, respectively.

21. The method of claim 13, the light propagates along a banana-shaped path, and the light attenuation equation is:
Iₒ/I=exp{Σᵢ₌₁^{M}((a1*Hbᵢ(x,y,z)+a2*HbOᵢ(x,y,z))*ΔLᵢ*r*ΔSᵢ)}
where Iₒ is the intensity of the incident light, I is the intensity of the reflected light, M means that the light propagation path is divided into M segments, with each segment being approximately a truncated cone, Hbᵢ(x,y,z) is the deoxyhemoglobin concentration of the i-th segment on the light propagation path, HbOᵢ(x,y,z) is the oxyhemoglobin concentration of the i-th segment on the light propagation path, a1 and a2 are the molar attenuation coefficients of deoxyhemoglobin and oxyhemoglobin, respectively, and ΔLᵢ is the height of the truncated cone for the i-th segment on the light propagation path, ΔS is the cross-section area of the i-th segment on the light propagation path in the middle, and r is a diffuse reflection path coefficient to be optimized through experiments.

22. The method of claim 14, deoxyhemoglobin and oxyhemoglobin concentration on light propagation path are interpolated from the deoxyhemoglobin and oxyhemoglobin concentration variables at the vertices of the finite elements through the finite element interpolation basis function.

23. A method for evaluating the blood circulation health level of human tissues, including:
controlling the light source on the hemoglobin sensor module to emit light of one or more frequencies and transmit the light waves to the patient's tissue;
controlling multiple light detectors at different positions to simultaneously detect the reflected light from human tissue and generating corresponding output signals;
determining the signal quality of the output signals and selecting high quality signals to perform tomographic reconstruction and calculate oxyhemoglobin and deoxyhemoglobin concentrations, and/or calculating pulse wave and oxygen saturation from each hemoglobin sensor module, and also calculating the oxygen saturation distribution and/or pulse wave propagation path based with the hemoglobin sensor module array positional information;
assessing the blood circulation health condition based on the concentration distribution of oxyhemoglobin, deoxyhemoglobin, pulse wave propagation pattern and/or the oxygen saturation map.
